(19) United States
(12) Patent Application Publication
(10) Pub. No.: US 2007/0021840 A1
(43) Pub. Date: Jan. 25, 2007

(54) Temporal stricture expander

(76) Inventor: Jorge Lopera, Dallas, TX (US)

Correspondence Address: PATENT DEPARTMENT;TAYLOR, PORTER, BROOKS & PHILLIPS, L.L.P, P.O. BOX 2471, BATON ROUGE, LA, 70821-2471, US

(21) Appl. No.: 11/185,332

(22) Filed: Jul. 20, 2005

(51) Int. Cl. A61F 2/04

(52) U.S. Cl. 623/23.7; 606/108

(57) ABSTRACT

An apparatus and method for treating strictures in an organ (e.g., a biliary duct or ureter) of a mammal by temporally expanding a collapsed or occluded fluid passageway in the anatomy to allow fluid (e.g., bile or urine) to flow through the passageway. The apparatus comprises a guider, a network of flexible metallic wires, and one or two retainers, depending on its use, to allow for the deployment and the retrieval of the apparatus. To temporarily expand a collapsed or occluded fluid passageway in the organ, the network of flexible metallic wires is compressed into an insertion form using an applicator and inserted into the body to a position within the collapsed or occluded passageway. The network of flexible metallic wires is then expanded into a larger operational form within the passageway to open and support the passageway.

This invention pertains to a retrievable, expandable device that can be implanted into the anatomy of a mammal to treat strictures in organs (e.g., a biliary duct) by temporally expanding the volume of the organ to form a passageway.

Biliary stricture is a common medical condition affecting many humans. It occurs when the bile duct becomes constricted, inhibiting bile from draining into the intestine. This causes the bile to back up into the liver and to spill over into the blood stream resulting in obstructive jaundice. There are many medical-related causes for biliary strictures. These causes may be benign (non-cancerous) or cancerous. Cancerous bile duct strictures are caused by bile duct cancer and pancreatic cancer. Benign biliary strictures often develop from symptoms related to chronic pancreatitis, injuries to the bile duct when performing procedures such as laparoscopic cholecystectomy, or other open surgical procedures, such as bilio-enteric anastomosis. Inflammatory diseases such as sclerosing cholangitis can also cause benign biliary strictures. Surgery is often the most effective means of treating benign biliary strictures.

Studies have shown that in the case of benign biliary strictures, surgical treatment has a four-year success rate in about 86 to about 88 percent of patients. However, biliary strictures that recur after surgery are often more difficult to treat, and thus result in a lower success rate. The success rate is also lower in patients who suffer from difficult access due to scar tissue from previous surgeries, or comorbid (i.e., two or more diseases which occur together) conditions.

There are several non-surgical procedures for treating benign biliary strictures. One procedure for treating benign biliary strictures is referred to as “percutaneous transhepatic balloon dilatation” of biliary strictures, and involves introducing a 22- to 23-gauge needle through the skin in the right or left intercostal space of the midaxillary line, and then advancing the needle into the liver parenchyma under fluoroscopic guidance. The biliary is then opacified by injecting contrast material into the liver parenchyma as the needle is slowly withdrawn. Next, a guidewire is passed through the stricture and then a dilatation balloon is used to dilate the stricture. Balloon dilatation of biliary strictures can also be performed using endoscopic techniques. However, this procedure is not always effective, and many patients often repeatedly require interventions and eventually surgery.

Another procedure for treating benign biliary strictures is referred to as “metallic stent placement” and involves placing a metallic stent into the stricture to expand the fluid passageway and to allow bile to flow through the bile duct. Metallic stents are considered to be a permanent implantable device because the occurrence of tissue proliferation in and around the struts of the metal stents makes them difficult or nearly impossible to remove. Patients undergoing this procedure to treat a biliary stricture have a high probability that the stricture will reoccur due to granulated tissue reaction and mucosal hypertrophy. The proliferation of tissues is a natural reaction of the body to metal and to the presence of a foreign body. In addition, strictures often re-form because the patency of the stents decreases to less than 50 percent within the first 12 to 18 months, and may decrease to as low as zero percent after five years. Chronic inflammation and obstruction caused by the metallic stents may predispose the patient to repeated episodes of cholangitis (i.e., infection in the bile ducts) and cholangiocarcinoma.

B. D. Petersen et al., “Treatment of Refractory Benign Biliary Stenoses in Liver Transplant Patients by Placement and Retrieval of a Temporary Stent-Graft: Work in Progress.” J. Vasc. Interv. Radiol., vol. 11(7), pp. 919-929 (2000) discloses a retrievable metallic stent-graft for use in some patients with benign bile duct strictures following liver transplant surgery. The device operates by placing the stent-graft into the benign bile duct stricture and opening the stricture by inflating the balloon. Once implanted, the stent-graft may be removed by inserting a guidewire and sheath having a balloon angioplasty catheter through the lumen of the stent-graft, inflating the balloon catheter, and then pushing the stent-graft and balloon catheter into an adjacent bowel. Next, the tip of the sheath is flared with the angioplasty catheter and the balloon deflated, so that it can be advanced into the stent-graft. The balloon catheter is then re-inflated to fix the stent-graft on the balloon catheter. A snare is then advanced over the balloon catheter and positioned around the most proximal portion of the stent-graft that is located in the bowel. The snare is then tightened around the stent-graft and the balloon catheter gradually deflated. The snare and stent-graft are then gradually drawn into the sheath for removal.

There are several complications associated with this procedure. For example, the balloon-expandable stent requires relatively large delivery and retrievable systems (e.g., 12-16 Fr catheters), which increases the risks of bleeding due to accidental injuries to the portal, hepatic veins, and hepatic artery, in addition to increasing pain and the risk of incurring infections. It has also been reported that the proximal suture broke on several occasions. Furthermore, in at least one instance, it was reported that when one strut of the metal stent remained outside the sheath during retrieval, the patient experienced a forceful, painful extraction through the liver resulting in a large portal vein laceration.

U.S. Pat. Pub. No. 2004/0127973 describes a device for preventing luminal occlusions and treating both benign and malignant biliary strictures, comprising a flexible, metallic scaffolding configured to define a substantially cylindrical member having a lumen, regions with different hardness and softness levels to improve patient comfort, and a plurality of eyelets with suture threaded there-through for removal of the device. In one embodiment, an installed device is removed from the anatomy of a patient by grasping the suture and pulling on the suture to reduce the outward radial force exerted by the device on the patient's anatomy.

U.S. Pat. No. 5,350,398 describes a device for filtering blood flowing through a fluid passageway in a living body, comprising a flexible stent and a first plurality of flexible wire segments attached to the stent. In one embodiment, the flexible stent is compressed into a first insertion form and percutaneously inserted into the passageway. The flexible stent is then expanded into a larger operational form within the passageway to open and support the passageway, and to allow the flexible wire segments to unbend and form a first filter element across the passageway. In a preferred embodiment, the stent additionally comprises barbs or hooks to help prevent migration by fixing the stent in the passageway.

An unfilled need exists for a retrievable, non-surgical instrument whose structure, once implanted into the anatomy of mammals, allows for the treatment of strictures in organs (e.g., the biliary duct and ureter) by temporarily expanding a collapsed or occluded fluid passageway in the organ to form a fluid passageway.

I have discovered a retrievable, non-surgical instrument and method for treating strictures in organs (e.g., the biliary duct) of a mammal by temporarily expanding a collapsed or occluded fluid passageway in the anatomy to allow fluid (e.g., bile) to flow through the passageway. The device is able to expand a collapsed or occluded fluid passageway for at least two months, while filtering the fluid flowing through the passageway. The device is a “temporal stricture expander” comprising a network of flexible metallic wires and a retainer to allow for the deployment and the retrieval of the device in the biliary duct. Preferably, the device further comprises a guider adapted to aid in the positioning of the network of flexible metallic wires.

Modifications may be made to the temporal stricture expander to treat strictures in other organs (e.g., the ureter) of a mammal by temporally expanding a collapsed or occluded fluid passageway in the anatomy to allow fluid (e.g., urine) to flow through the passageway. In this embodiment, the device comprises a network of flexible metallic wires and two retainers to allow for the deployment, stabilization, and the retrieval of the device in the urinary system.

Both designs allow for the temporal expansion of a collapsed or occluded fluid passageway by compressing the network of flexible metallic wires into an insertion form using an applicator (e.g., a peel-away introducer sheath), and inserting the network of flexible metallic wires into the body to a position within the collapsed or occluded passageway. The network of flexible metallic wires may then be expanded into a larger operational form within the passageway to open and support the passageway, and to allow a portion of the network of flexible metallic wires to decompress and form a filter element leading into the passageway. The device may be removed from the passageway by compressing the network of flexible metallic wires back into their insertion form and gently pulling the retainer until the network of flexible metallic wires exits the anatomy. In a preferred embodiment, the retainers comprise a perforated section with holes sized to withdraw at least a portion of fluid (e.g., bile or urine) from the body of the mammal.

### BRIEF DESCRIPTION OF THE FIGURES

FIG. 1 illustrates a side plan view of one embodiment of the temporal stricture expander with an applicator.

FIG. 2 illustrates a schematic view of another embodiment of the temporal stricture expander without an applicator.

FIG. 3 illustrates a side plan view of the temporal stricture expander in FIG. 2 with a “pig-tailed” end.

The invention provides reliable surgical devices and methods for temporally expanding collapsed or occluded fluid passageways in organs (e.g., the biliary duct and ureter) of a mammal to form a fluid passageway. Candidates for these minimally invasive, non-surgical procedures include patients who cannot undergo surgery because of the presence of conditions such as jaundice and cholangitis caused by benign biliary strictures, or benign ureteral strictures that may cause hydronephrosis and kidney damage, and patients who have unsuccessfully undergone balloon dilatation to treat these strictures. In one embodiment, the device is a temporal stricture expander comprising a network of flexible metallic wires and a retainer to allow for the deployment and the retrieval of the device in biliary ducts. Preferably, the device further comprises a guider adapted to aid in the positioning of the metallic stent. The guider is located inside the retainer and extends past the network of flexible metallic wires to guide the network of flexible metallic wires into the anatomy.

A standard percutaneous biliary drainage procedure is used to facilitate the deployment and retrieval of this device from the bile duct. To achieve this, the right or left biliary-duct is accessed and the guider is advanced across the stricture. In cases of severe stenosis, the stricture is dilated with an 8-10 mm balloon. The network of flexible metallic wires is compressed into an insertion form by advancing an applicator (e.g., a peel-away introducer sheath) over the retainer and the network of flexible metallic wires until the stent is completely drawn into the applicator. The applicator is then advanced over the guide wire and across the stricture. The guide wire is used to advance the device into the desired location inside the biliary or urinary system, using an inner hole or conduit that allows the stent to be advanced over the guide wire.

In another embodiment, the temporal stricture expander comprises a network of flexible metallic wires having a distal end, a proximal end, a protective layer, and a first and second retainer attached to the ends of the network of flexible metallic wires for deployment in the urinary system (e.g., the ureter). In this embodiment, the retainers are adapted to allow the center of the network of flexible metallic wires to expand, while the proximal and distal ends are collapsed inside the retainers for placement in the urinary system. A standard percutaneous urinary drainage procedure, also known as percutaneous nephrostomy, is used to facilitate the deployment and retrieval of the device from the kidney or the urinary bladder. The network of flexible metallic wires is compressed into an insertion form by advancing an applicator (e.g., a peel-away introducer sheath) over the first retainer, the network of flexible metallic wires, and then the second retainer. The applicator is then advanced over a guide wire and across the stricture. In a preferred embodiment, the second retainer is adapted to form a “flexible pigtail” capable of curling to form a movement barrier in the bladder to prevent post-implantation movement of the temporal stricture expander.

In both devices, once the network of flexible metallic wires is centered at the stricture, it is deployed under fluoroscopic control by slowly removing the applicator while the network of flexible metallic wires and retainer are held stationary. Contrast material is injected through an external adapter attached to the retainer to verify that the distal end of the network of flexible metallic wires (i.e., the portion covered with a protective layer made from a membrane such as polyurethane, silicone, or PERMALUME®) is placed across the stricture, while the proximal end of the network of flexible metallic wires (i.e., the non-covered portion of the network of flexible metallic wires) is placed just above the stricture. Preferably, the retainer comprises a perforated section with drain ports sized to allow for at least a portion of the fluid near the stricture to be withdrawn from the body and for the injection of a saline solution near the stricture. To prevent post-implantation movement of the temporal stricture expander, the external portion of the retainer may be fixed to the skin of the patient using sutures. The position of the network of flexible metallic wires may be checked periodically by injecting contrast medium through the retainer to confirm that the network of flexible metallic wires is positioned properly. If the network of flexible metallic wires becomes dislodged from the stricture, it can be repositioned by slowly and gently manipulating the retainer.

The temporal stricture expander can be removed from the passageway by re-advancing the applicator over the retainer and the network of flexible metallic wires until the network of flexible metallic wires is completely drawn into the applicator, and then slowly and gently pulling the applicator and network of flexible metallic wires out of the patient's body.

There are several advantages of using the novel devices to expand a collapsed or occluded fluid passageway in the anatomy of a patient. First, the number of components may be minimal. Fabrication may be simple and inexpensive. Second, the devices are able to use metallic stents having a large luminal diameter and improved patency. Third, the network of flexible metallic wires can easily be removed from a patient after more than two months of initial placement without causing traumatic injury to surrounding tissues and organs because the system performs as if it were a network of flexible metallic wires that was only partially deployed. Fourth, drain ports in the retainer allow for the external drainage of fluid (e.g., bile or urine) in cases of infection or occlusion of the network of flexible metallic wires.

### EXAMPLE 1

Temporal Stricture Expander Design for use in a Biliary System

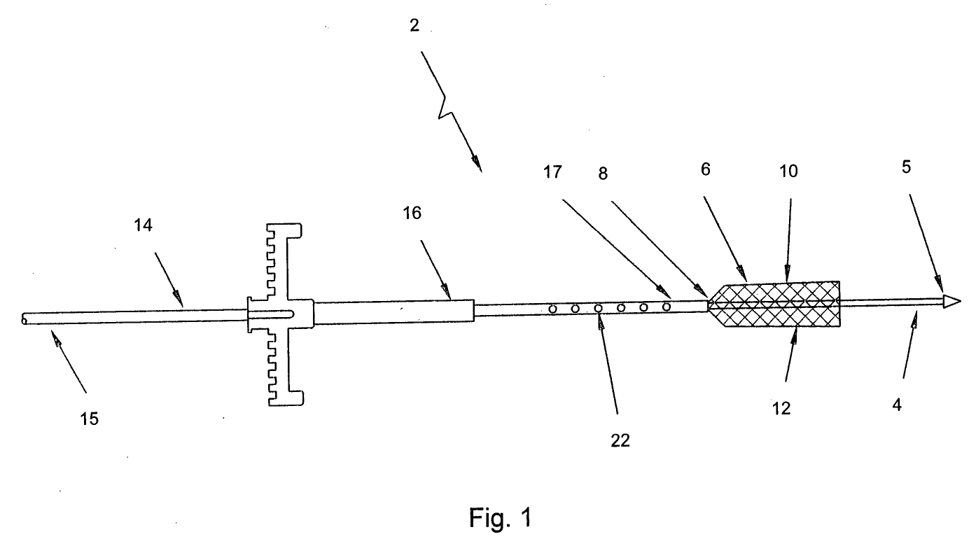

FIG. 1 illustrates a side plan view of one embodiment of the biliary temporal stricture expander 2 in accordance with this invention. In this embodiment, temporal stricture expander 2 comprises a guider 4 having a tip 5, a network of flexible metallic wires 6 having a proximal end 8 and a distal end 10, and a retainer 14 attached to proximal end 8 to allow for the deployment and the retrieval of the device. Network 6 was sized and shaped to expand a collapsed or occluded fluid passageway in a biliary duct, when positioned across a stricture, and was adapted to allow for compression into an insertion form (i.e., a compressed form of the network of flexible metallic wires that allows it to be inserted into a stricture) for deployment such as a WALLSTENT® (Microvasive, Boston Scientific, Natick, Mass.). To compress network 6, an applicator 16 was guided over retainer 14 to a position near network 6, and network 6 was gently pulled into applicator 16 with retainer 14. In this embodiment, applicator 16 was a peel-away introducer sheath (Boston Scientific, Natick, Mass.) sized and shaped to complement that of retainer 14 and network 6 such that applicator 16 was able to be advanced over retainer 14, and to compress network 6 into the insertion form. Network 6 was also able to inhibit mucosa and granulation tissue from growing through wire segments 12 by shielding the middle segment or body and distal end of network 6 from surrounding tissue in the biliary duct using a protective membrane (not shown; e.g., polyurethane, silicone or PERMALUME®).

As shown in the FIG. 1, guider 4 comprises a catheter having a proximal end (not shown) and a distal end 18 with a flared, tapered tip 5. In this embodiment, guider 4 was sized and shaped to complement that of retainer 14 and network 6 such that guider 4 was able to be advanced through network 6. Tapered tip 5 was sized and shaped to complement the size of applicator 16 such that when applicator 16 was advanced over guider 4, guider 4 formed the end of applicator 16 and helped guide network 6 into a stricture within the organ (e.g., a biliary duct) of the patient.

As shown in FIG. 1, retainer 14 comprises a proximal end 15 and a distal end 17. In this embodiment, retainer 14 was a catheter sized and shaped to complement that of network 6 such that distal end 17 of retainer 14 was able to be permanently attached to proximal end 8 of network 6 and to allow for the insertion and retrieval of network 6. In a preferred embodiment, distal end 17 of retainer 14 comprises a perforated section with drain ports 22 sized to allow for at least a portion of fluid near the stricture to be withdrawn from the body and for the injection of a saline solution irrigation near the stricture. The position of network 6 may be checked periodically by injecting contrast medium through retainer 14 to confirm that metallic it is positioned properly. If network 6 becomes dislodged from the stricture, it can be repositioned by slowly and gently manipulating retainer 14.

### EXAMPLE 2

Temporal Stricture Expander Design for use in a Urinary System

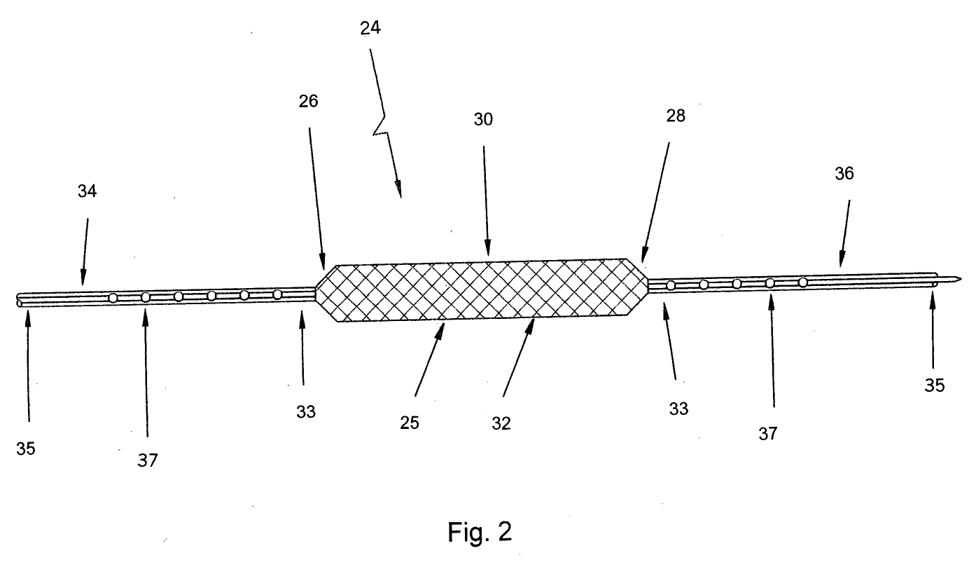

FIG. 2 illustrates a schematic view of one embodiment of the urinary temporal stricture expander 24 in accordance with this invention. In this embodiment, temporal stricture expander 24 comprises a network of flexible metallic wires 25 having a proximal end 26 a distal end 28, and a center section 30, a guider 31, and a first retainer 34 and second retainer 36 to allow for the deployment, anchoring, and retrieval of the device in a urinary system.

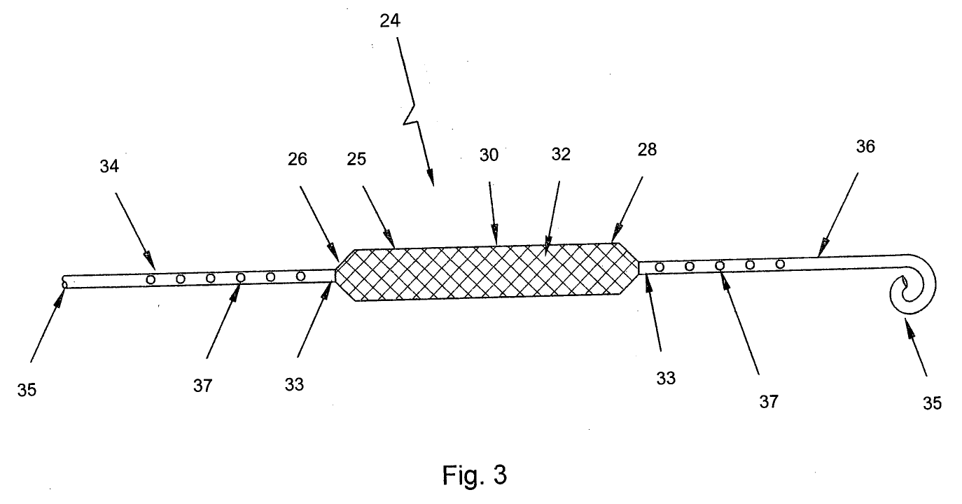

As shown in FIG. 2, first and second retainers 34 and 36, respectively, each comprise a distal end 33 and a proximal end 35 having perforated sections with drain ports 37 sized to allow for a portion of fluid near a stricture to be withdrawn from the body and for the injection of a saline solution irrigation near the stricture. In a preferred embodiment, second retainer 36 further comprises a distal end 37 adapted to form a flexible “pigtail design” able to stabilize the device when placed in the bladder. See FIG. 3. As previously explained in Example 1, an applicator (not shown; e.g., a peel-away introducer sheath manufactured by Boston Scientific, Natick, Mass.) was sized and shaped to complement that of retainers (34 and 36) and network 25 such that applicator 16 was able to be advanced over first retainer 34, to compress a network 25 into an insertion form, and to be advanced over second retainer 36. See item 16, FIG. 1. Network 25 was also able to inhibit mucosa and granulation tissue from growing through wire segments 32 by shielding center section 30 of network 25 from surrounding tissue in the ureter, using a protective membrane (not shown; e.g., polyurethane, silicone, or PERMALUME®).

As shown in FIG. 2, guider 31 was sized and shaped to complement that of retainers 34 and 36 and network 24 such that guider 31 was able to be advanced through network 24 and retainer 36 to help guide network 24 into a stricture within the organ (e.g., a ureter) of the patient.

### EXAMPLE 3

Construction of the Prototype Biliary, Temporal Stricture Expander for Use in the Biliary System

A prototype biliary, temporal stricture expander 2 comprising a guider 4, a network 6 having a proximal end 8, a distal end 10, and a network of flexible metallic wires 6, and a retainer 14 to allow for the deployment and retrieval of the device in a biliary system, as shown in FIG. 1, was constructed. Guider 4 was constructed from a 4 Fr polytetrafluoroethylene inner tubing having a 0.035 in inner diameter (Small Parts, Miami Lakes, Fla.) that form a channel for passage of a guide wire. The enclosed tip 5 of guider 4 was tapered to form an end to applicator 16, and to penetrate the stricture and any soft tissue near the stricture.

Retainer 14 was fabricated from an 8-10 Fr biliary tube (Boston Scientific, Natick, Mass.) made of polyurethane. An adapter (not shown) was attached to the proximal end 15 of retainer 14 (i.e., the end of the retainer which is located outside of a patient) to allow for the injection of contrast medium into the bile duct, and to irrigate the bile duct with a saline solution. The adapter also allowed for the connection of a drainage bag to drain any bile withdrawn from the bile duct. Holes 22 having a diameter of 2 mm were drilled through distal end 17 of retainer 14 to form drain ports able to receive a saline solution to flush the stricture or a contrast medium to verify stent patency.

Network 6 was fabricated from a biliary a WALLSTENT® (Microvasive, Boston Scientific, Natick, Mass.) partially encased in PERMALUME® and had a length of 8 cm and a diameter of 10 mm. Network 6 had a 5 mm long uncovered portion at both the proximal and distal ends (8 and 10). Network 6 was cut in half such that the distal end 10 was fully covered by a protective membrane and the proximal end 8, which was 5 mm long, was uncovered. A portion of the proximal end 8 (˜2.5 mm) was compressed to an insertion form, inserted over guider 4, and then partially advanced into the distal end 17 of retainer 14. This portion was then glued to retainer 14 using medical grade cyanocrylate. Approximately 2.5 mm of proximal end 8 of network 6 remained outside of retainer 14 to form a filtered inlet to the fluid passageway.

### EXAMPLE 4

Construction of the Prototype Urinary, Temporal Stricture Expander for Use in the Urinary System

A prototype urinary, temporal stricture expander 24 comprising a network of flexible metallic wires 25 having a proximal end 26, a distal end 28, a center section 30 and wire segments 32, a guider 31, and a first retainer 34 and second retainer 36 to allow for the deployment and retrieval of the device in a urinary system, as shown in FIG. 2, was constructed. Second retainer 36 further comprises a distal end 37 adapted to form a flexible “pigtail design”-able to stabilize the device when placed in the gall bladder.

Retainers (34 and 36) were fabricated from an 8-10 Fr biliary tube (Boston Scientific, Natick, Mass.) made of polyurethane. An adapter (not shown) was attached to first retainer 34 (i.e., the end of the retainer which is located outside of a patient) to allow for the injection of contrast medium into the urinary system and to irrigate the urinary system with saline solution. The adapter also allowed for the connection of a drainage bag to drain any urine withdrawn from the urinary system. Holes 22 having a diameter of 2 mm were drilled through a distal portion of retainers (34 and 36) to form drain ports able to receive a saline solution to flush the stricture or a contrast medium to verify stent patency.

Network 25 was fabricated from a WALLSTENT® (Microvasive, Boston Scientific, Natick, Mass.) partially encased in PERMALUME® and had a length of 4.0 cm and a diameter of 8-10 mm. Network 25 had a 5 mm long uncovered portion at both the proximal and distal ends (26 and 28, respectively). A portion (˜2.5 mm) of the proximal and distal ends (26 and 28, respectively) was compressed to an insertion form, and then partially advanced into the proximal ends 33 of retainers (34 and 36). These portions were then glued to retainers (34 and 36) using medical grade cyanocrylate. Approximately 2.5 mm of proximal and distal ends (26 and 28) of network 25 remained outside of retainers (34 and 36) to form filtered inlet and outlets to the fluid passageway.

### EXAMPLE 5

Testing of the Prototype Biliary, Temporal Stricture Expander

To confirm that the prototype biliary, temporal stricture expander 2 was highly effective in temporally expanding a collapsed or occluded fluid passageway in a biliary duct of a mammal, tests were performed on dogs to treat strictures located in the bile duct using the prototype of Example 3.

The patient was first prepared by checking blood coagulation parameters, and then by administering broad-spectrum prophylactic antibiotics at least one hour before the procedure. The patient was then placed on an angiographic table on its back for prepping and draping in a sterile fashion, and sedated with an intravenous anesthetic (e.g., Fentanyl and Versed). Vital signs, blood pressure and oxygen saturation were checked continuously during the procedure. A percutaneous cholangiogram (i.e., an x-ray examination of the bile ducts and areas inside the liver) was performed on the patient by injecting a contrast medium into the biliary ducts using a 21-guage Chiba needle (Boston Scientific, Natick, Mass.). Once the biliary ducts were opacified, a 0.018-inch wire was advanced into the central bile ducts. A coaxial 6 Fr system (Accustick, Boston Scientific, Natick, Mass.) was then used to advance a 0.035-inch guide wire 4 across the stricture. Next, the tract in the liver parenchyma was dilated to 10 Fr using vessel dilators (Coons dilator, Cook, Bloomington Ind.) and the 10 Fr internal/external biliary drainage catheter was inserted into the liver parenchyma, with the distal tip of the drain catheter positioned in the duodenum and the proximal end above the stricture. The external portion of the catheter was then sutured to the skin of the patient and connected to a drainage bag for external drainage of bile. The patient was prescribed an oral, broad-spectrum antibiotic to inhibit the occurrence of infection.

The patient returned after the tract matured and the risks of bile leakage and bleeding from subsequent manipulation of a network of flexible metallic wires 6 decreased. The average time for tract maturity following surgery was approximately 10-15 days. The existing biliary drainage tube was prepped and draped in a sterile fashion. Contrast material was injected through the end of the tube to verify the location of the stricture. A 0.035-inch guide wire 4 was then advanced through the biliary tube and the tube was removed after the sutures were cut. A local anesthesia was then administered in the skin and subcutaneous tissues of the patient. In cases of severe stenosis, the stricture is dilated with an 8-10 mm balloon.

Once the local anesthesia was administered, the network of flexible metallic wires 6 was then compressed into an insertion form by advancing an applicator 16 (e.g., a 10 Fr peel-away introducer; Boston Scientific, Natick, Mass.) over the retainer 14 until the network of flexible metallic wires 6 was completely drawn into the applicator 16. Next, the applicator 16 was advanced over the guide wire 4 and across the stricture, and then the network of flexible metallic wires 6 and retainer 14 were advanced as a unit over the guide wire 4 until the network of flexible metallic wires 6 reached the center of the stricture. The network of flexible metallic wires 6 was then deployed into the stricture under fluoroscopic control by slowly withdrawing the applicator 16 from the stricture while the rest of the apparatus remained stationary. The applicator 16 was then completely removed from the retainer 14.

Once the applicator 14 was removed, Iohexol (Omnipaque, Amersham Health, Princeton, N.J.) was then injected into the stricture via an external adapter to verify that the main body of the network of flexible metallic wires 6 was correctly positioned across the stricture while the proximal end 8 (i.e., the non-covered section of the stent) was located just above the stricture. The external portion of the retainer 14 was then fixed to the patient's skin using suture (2-0 Prolene suture; Ethicon, Somerville, N.J.). The position of the network of flexible metallic wires 6 was checked periodically by injecting contrast medium through the external adapter attached to the retainer 14 to confirm that the network of flexible metallic wires 6 was positioned properly. (If the network of flexible metallic wires 6 is dislodged from the stricture, it can be repositioned by slowly and gently manipulating the retainer 14.)

The temporal stricture expander 2 was removed from the patient after approximately two months by re-advancing the applicator 16 over the retainer 14 and the network of flexible metallic wires 6 until the network of flexible metallic wires 6 collapsed and was completely drawn into the applicator 16. The applicator 16 and network of flexible metallic wires 6 were then slowly and gently pulled out of the patient's body as a unit. A cholangiogram was then performed to verify that the passageway in the bile duct remained open.

From the above animal and in vitro tests, several conclusions were reached. The temporal stricture expander 2 was effective in treating strictures in a bile duct by temporally expanding the occluded fluid passageway and allowing bile to flow through the passageway. The retainer 14 produced at least two beneficial outcomes. First, a portion of the bile near the network of flexible metallic wires 6 was withdrawn from the patient to help improve the liver function by decompressing the liver and decreasing the risks of infection. Also the external portion allows for the injection of contrast to verify patency and correct positioning of the network of flexible metallic wires 6. Second, the retainer 14 was effective in helping to insert and to remove the network of flexible metallic wires 6, and minimized the affects of granulation tissue reaction and injury to the bile duct.

### EXAMPLE 6

Testing of the Prototype Urinary, Temporal Stricture Expander

To confirm that the prototype urinary, temporal stricture expander 24 is highly effective in temporally expanding a collapsed or occluded fluid passageway in a ureter of a mammal, tests will be performed on five mongrel dogs to treat strictures located in the ureter, using the prototype of Example 4. A model of ureteral stricture will then be created by placing an elastic band around the mid portion of the ureter about a week later. Once the kidney develops hydronephrosis (i.e., urinary retention), a percutaneous nephrostomy will be performed, and then the urinary, temporal stricture expander 24 will be deployed in the area of stricture. Follow-up nephrostograms with injections of dye will be performed on a weekly basis. After the fourth week the network of flexible metallic wires 25 will be removed and two animals will be sacrificed immediately for histological analysis. The remaining three animals will be sacrificed two weeks later for histological analysis and comparison.

In the future, additional tests will be conducted to determine the most effective time in which the network of flexible metallic wires should be removed to allow the healing process to be completed, and to verify that the apparatus does not create any additional injuries to the biliary or ureter system.

The complete disclosures of all references cited in this specification are hereby incorporated by reference. In the event of an otherwise irreconcilable conflict, however, the present specification shall control.

I claim:

1. A device for temporary placement within a mammal at a position near an organ for the treatment of a collapsed or occluded fluid passageway within the organ; said device comprising:
(a) a network of flexible metallic wires having a proximal end and a distal end; wherein said network is sized and shaped such that when said network is compressed into a smaller first shape, said network can be inserted across the collapsed or occluded fluid passageway, and when said network is allowed to expand into a larger second shape, a collapsed or occluded fluid passageway can be expanded to a size larger than the size of the collapsed or occluded fluid passageway before said network is inserted; and
(b) one or two retainers, each having a proximal end and a distal end; wherein said distal end of said one or two retainers is attached to said network to allow for the deployment, anchoring, and the retrieval of said network;
wherein:
(c) said device is adapted to be placed within a mammal at a position near an organ for the treatment of a collapsed or occluded fluid passageway within the organ, and then to be removed after a period of about two months or longer.

2. A device as recited in claim 1, wherein said device further comprises a guider adapted to extend pass said network and to guide said network through the collapsed or occluded fluid passageway.

3. A device as recited in claim 1, wherein said device further comprises an applicator; wherein said applicator is sized and shaped to complement the size and shape of said one or two retainers and said network such that when said applicator is advanced over said network, said network is compressed into said smaller first shape; and wherein when said applicator is removed from said network, said network expands into said larger second shape; whereby said device is prepared for temporary deployment into the collapsed or occluded fluid passageway by advancing said applicator over said one or two retainers and said network to compress said network into said smaller first shape; and whereby said network is retrieved from a collapsed or occluded fluid passageway by advancing said applicator over said one or two retainers and said network to compress said network into said smaller first shape, and then light pressure is applied to one of said one or two retainers to pull said device out of the organ; and wherein said device is advanced into the organ until at least a portion of said network is positioned across the collapsed or occluded fluid passageway, and then said applicator is removed from said one or two retainers and said network to expand and support the passageway, and to allow more fluid to flow through the expanded fluid passageway than the amount of fluid flowing through the collapsed or occluded passageway.

4. A device as recited in claim 3, wherein at least a portion of said network comprises a protective-layer cover.

5. A device as recited in claim 4, wherein said protective layer cover is selected from the group consisting of polyurethane and silicone.

6. A device as recited in claim 1, wherein the organ is a biliary duct.

7. A device as recited in claim 1, wherein said distal end of at least one of said one or two retainers additionally comprises a perforated section with holes sized and shaped such that at least a portion of fluid in the organ may be withdrawn from the passageway or a saline solution may be injected into the passageway.

8. A device as recited in claim 4, wherein said portion of said network is adapted to form a filter element for filtering at least a portion of fluid flowing through the expanded passageway.

9. A device as recited in claim 1, wherein the organ is a ureter.

10. A method of treating a collapsed or occluded fluid passageway in an organ of a mammal using a device as recited in claim 1; said method comprises inserting the applicator containing the network of flexible metallic wires, guider, and one or two retainers to a predetermined position near the organ; advancing the distal end of the applicator into the collapsed or occluded passageway; and removing the applicator from the organ while the network of flexible metallic wires and the one or two retainers are held stationary with the distal end of the network of flexible metallic wires located in the collapsed or occluded fluid passageway.

11. A method as recited in claim 10, wherein the organ is a biliary duct.

12. A method as recited in claim 10, wherein the organ is a ureter.

13. A method as recited in claim 10, wherein the distal end of at least one of the one or two retainers additionally comprises a perforated section with holes sized and shaped such that a portion of the fluid in the organ may be withdrawn from the passageway or a saline solution may be injected into the passageway.

14. A method as recited in claim 13, wherein said method additionally comprises draining from the organ a portion of fluid in the vicinity of the network of flexible metallic wires.

15. A method as recited in claim 13, wherein said method additionally comprises injecting a saline solution in the vicinity of the network of flexible metallic wires to remove debris and help facilitate patency.

16. A method as recited in claim 10, wherein a portion of the network of flexible metallic wires comprises a protective-layer cover.

17. A method as recited in claim 16, wherein the protective layer cover is selected from the group consisting of polyurethane and silicone.

18. A method as recited in claim 10, wherein the proximal end of the network of flexible metallic wires is adapted to form a filter element for filtering at least a portion of fluid flowing through the expanded passageway.

19. A method as recited in claim 10, wherein the method additionally comprises attaching the proximal end of at least one of the one or more retainers to the skin of the patient using sutures.
